(19) United States
(12) Patent Application Publication
(10) Pub. No.: US 2020/0078286 A1
(43) Pub. Date: Mar. 12, 2020

(54) HAIR TREATMENT INTERMEDIATES AND METHODS

(71) Applicant: Tru-Hair LLC, Carlsbad, CA (US)

(72) Inventors: Jane M. Meyer, Carlsbad, CA (US); Logan George Jackson, Los Angeles, CA (US)

(21) Appl. No.: 16/496,847

(22) PCT Filed: Mar. 23, 2018
(86) PCT No.: PCT/US18/23999

§ 371 Date: Sep. 23, 2019

Related U.S. Application Data
(60) Provisional application No. 62/475,817, filed on Mar. 23, 2017.

(51) Int. Cl. A61K 8/65; A61K 8/73; A61K 8/86; A61K 8/22; A61K 8/41; A61Q 5/04; A61Q 5/10

(52) U.S. Cl. CPC A61K 8/65; A61K 8/73; A61K 8/86; A61K 8/22; A61K 8/411; A61K 2800/884; A61Q 5/10; A61K 2800/4324; A61K 2800/57; A61K 2800/59; A61Q 5/04

(57) ABSTRACT

Methods for improving a hair quality in a subject comprise mixing a polypeptide covalently bound to a hydrophilic polymer and/or a polysaccharide covalently bound to a hydrophilic polymer and an oxidizing agent, a reducing agent, or ammonia; and applying the composition to hair. Related compositions are also disclosed.

### CROSS-REFERENCE TO RELATED APPLICATION

This application claims the benefit of U.S. Application Ser. No. 62/475,817, filed Mar. 23, 2017. The disclosure of the prior application is considered part of and is incorporated by reference in the disclosure of this application.

### BACKGROUND

U.S. patent application Ser. No. 14/419,283 assigned to same assignees relates to compositions and methods for hair improvement. WO2015/187951 relates to compositions and methods for improving skin quality. Both are incorporated herein by reference in their entireties.

Various hair processes may be used to color or change the texture of hair, such as by bleaching, dyeing or using permanent waving (perming) or straightening solutions and products, which are well known in the industry. During these processes, hair may become over processed or damaged in part due to the harsh chemicals needed to effect the change desired. Many processes use oxidizing agents that can damage hair. In addition, hair can become heat damaged by applying heat to hair too frequently or at too high a temperature. It would be useful to the art to be able to prevent or ameliorate damage to hair while effecting change to the hair. It would also be useful to prevent or ameliorate damage without the use of heat. It would also be helpful to be able to apply a hair treating product at the same time as methods to color or change the texture of hair, and have the hair quality improve without the need for any additional steps.

There is a need, therefore, to develop compositions and methods for the protection of healthy hair or the amelioration of damaged hair.

### SUMMARY

Provided herein are compositions and methods that solve the problems or inadequacies of the prior art. Other features and advantages will be apparent from the following detailed description and the claims.

In some aspects, a hair treatment method comprises a) breaking a disulfide bond in a subject's hair to form a sulfide group on hair; b) binding the sulfide group on hair with a sulfide group on a polypeptide such as a hydrolyzed keratin polypeptide in a conjugate, wherein the polypeptide is covalently bound to a hydrophilic polymer such as a polyethylene glycol compound in the conjugate.

In some aspects, a hair treatment method comprises binding a conjugate to the subject's hair, wherein polysaccharide or polypeptide such as a hydrolyzed keratin polypeptide is covalently bound to a hydrophilic polymer such as a polyethylene glycol compound in the conjugate, without the use of heat.

In some aspects, a hair treatment method comprises mixing a) a conjugate comprising a polysaccharide or a polypeptide such as a hydrolyzed keratin polypeptide that covalently binds to hydrophilic polymer such as a polyethylene glycol to form the conjugate, and b) an oxidizing agent; to form a mixture; and applying the mixture to a subject's hair.

In some aspects, the applying step occurs after a reducing agent is applied to a subject's hair.

In some aspects, the oxidizing agent comprises hydrogen peroxide or is a neutralizing agent that neutralizes the reducing agent.

In some aspects, the mixture further comprises a dye or comprises a reducing agent.

In some aspects, the conjugate binds to hair without the use of heat.

In some aspects, a hair treatment method comprises mixing a) a conjugate comprising a polysaccharide or a polypeptide such as a hydrolyzed keratin polypeptide that covalently binds to hydrophilic polymer such as a polyethylene glycol to form the conjugate, and b) a reducing agent; to form a mixture; and applying the mixture to a subject's hair.

In some aspects, a hair treatment method comprises mixing a) a conjugate comprising a polysaccharide or a polypeptide such as a hydrolyzed keratin polypeptide that covalently binds to hydrophilic polymer such as a polyethylene glycol to form the conjugate, and b) ammonia; to form a mixture; and applying the mixture to a subject's hair.

In some aspects, a hair treatment intermediate comprises a p-diamine or p-aminophenol that covalently binds through an amine group to a conjugate, wherein the conjugate is comprises a polysaccharide or a polypeptide such as a hydrolyzed keratin polypeptide that covalently binds to a hydrophilic polymer such as a polyethylene glycol to form the conjugate.

In some aspects, a hair treatment combination comprises a conjugate comprising a polysaccharide or a polypeptide such as a hydrolyzed keratin polypeptide that covalently binds to hydrophilic polymer such as a polyethylene glycol to form the conjugate, and ammonia.

In some aspects, a hair treatment combination comprises a conjugate comprising a polysaccharide or a polypeptide such as a hydrolyzed keratin polypeptide that covalently binds to hydrophilic polymer such as a polyethylene glycol to form the conjugate, and a reducing agent.

In some aspects, a hair treatment combination comprises a conjugate comprising a polysaccharide or a polypeptide such as a hydrolyzed keratin polypeptide that covalently binds to hydrophilic polymer such as a polyethylene glycol to form the conjugate, and an oxidizing agent.

### DETAILED DESCRIPTION

Provided herein are compositions and methods for hair modification, and more particularly, compositions and methods that improve hair physical characteristics such as by increasing hair smoothness, strength, volume/thickness, and/or flexibility, including on a semipermanent basis.

Terms used throughout this application are to be construed with ordinary and typical meaning to those of ordinary skill in the art. However, Applicants desire that the following terms be given the particular definition as defined below.

As used in the specification and claims, the singular form “a”, “an,” and “the” include plural references unless the context clearly dictates otherwise. For example, the term “a cell” includes a plurality of cells, including mixtures thereof.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the term “carrier” encompasses any excipient, diluent, filler, salt, buffer, stabilizer, solubilizer, lipid, stabilizer, or other material well-known in the art for use in hair care formulations. The choice of a carrier for use in a composition will depend upon the intended application of the combination. In some embodiments, the hair improvement compositions include one or more preservatives. Suitable examples include, without limitation, phenoxyethanol, citric acid, potassium sorbate, and caprylyl glycol.

A “composition” is intended to include a combination of active agent and another compound or composition, inert (for example, a carrier or excipient such as water) or active. A “composition” may also include a “combination,” which may refer to two or more active agents, such as the conjugate herein and the oxidizing agent, reducing agent, or ammonia, or a composition that contains an oxidizing agent, reducing agent, or ammonia, such as in hair processes such as bleaching, dyeing, perming or straightening compositions.

A “conjugate” may refer to a polypeptide covalently bound to a hydrophilic polymer and/or a polysaccharide covalently bound to a hydrophilic polymer, such as those described in U.S. patent application Ser. No. 14/419,283 and WO2015/187951, which are incorporated herein by reference in their entireties.

The term “covalently bound” means that a covalent bond exists between a portion of the hydrophilic polymer and the polysaccharide or polypeptide, or vice-versa. Accordingly, the term “covalently bound” refers to both direct covalent bonds (a sharing of pairs of electrons between the atoms of the hydrophilic polymer and the polypeptide) and indirect covalent bonds (a sharing of pairs of electrons between atoms of the hydrophilic polymer and the composition comprising the polypeptide, or vice-versa).

An “effective amount” is an amount sufficient to effect beneficial or desired results. An effective amount can be administered in one or more administrations, applications or dosages.

The methods provided herein may be used to achieve multiple hair improvement effects. As used herein, the term “hair improvement effect” includes, but is not limited to, smoothing, thickening, repairing, increased flexibility, and coating. Hair smoothing may be measured using any method known to one of skill in the art, including but not limited to, scanning electron microscopy (SEM) of the hair shaft, and the methods described in U.S. Pat. No. 8,151,624. When using SEM, hair smoothing may be indicated by an increase in either the number of tight junctions between the hair cuticle plates or a decrease in the degree of openness in the junctions between the hair cuticle plates. Hair thickening is defined herein as an increase in hair shaft diameter by greater than 15 microns. In some embodiments, hair thickening is an increase in hair shaft diameter by 16-80 microns, 20-70 microns, 25-50 microns, or 30-40 microns. Increased flexibility of the hair may be measured by a decrease in hair breakage, by an increase in tensile strength, or any other method known to those of skill in the art. Methods of measuring tensile strength are known to those of skill in the art and can take into account Hooke's Law, Young's modulus (or the module of elasticity), yield strength, alternate moduli, and strain.

“Mammal” for purposes of treatment refers to any animal classified as a mammal, including human, domestic and farm animals, nonhuman primates, and zoo, sports, or pet animals, such as dogs, horses, cats, cows, etc.

An “oxidizing agent” may refer to a developer used in a permanent, demi-permanent or semi-permanent hair color or a permanent curl or straightening composition that reacts with an alkaline agent (reducing agent) such as sodium hydroxide (straightening), or ammonia, ethanolamine, or sodium carbonate (coloring). Examples of oxidizing agents include hydrogen peroxide.

The term “semi-permanent” is defined herein to mean an effect existing beyond 10 hair washings or existing longer than one week following application of a hair improvement composition to the hair. In some embodiments, a semi-permanent effect exists after 25, 50, 100, or 200 hair washings following the application of the hair improvement composition to the hair. In other or further embodiments, a semi-permanent effect exists after 7, 14, 30, 60, 90, 120 or 180 days after the application of the hair improvement composition to the hair.

Accordingly, provided herein is a method of improving one or more physical characteristics of hair comprising contacting a least a portion of a subject's hair with a composition comprising a hydrophilic polymer-polypeptide conjugate or a hydrophilic polymer-polysaccharide conjugate, or both and an oxidizing agent.

The term “subject” is defined herein to include animals. In some embodiments, the animal is a mammal, including, but not limited to, primates (e.g., humans), cows, sheep, goats, horses, dogs, cats, rabbits, rats, mice and the like. In some embodiments, the subject is a human. In some embodiments, the subject is a child.

### DETAILED DESCRIPTION

Provided herein are methods for improving a hair quality in a subject comprising applying to the hair a composition comprising a polypeptide covalently bound to a hydrophilic polymer and/or a polysaccharide covalently bound to a hydrophilic polymer and an oxidizing agent, such as those found in bleach, hair dye, or permanent neutralizing solution, used for permanent waving (perming) or straightening hair. The polypeptides bound to the hydrophilic polymer include, but are not limited to, polypeptides of keratin, collagen, elastin, fibroin, milk-derived proteins such as casein, beta-lactoglobulin and alpha-lactalbumin, and grain proteins such as wheat protein. Hydrophobic polypeptides such as keratin may be preferred in certain embodiments. Hydrolyzed keratin polypeptides may be typically used in a conjugate. Also provided herein are compositions comprising a hydrophilic polymer covalently bound to a polysaccharide such as chitosan, chitin, or chitin glucan.

It is a surprising finding of the present invention that a composition containing a keratin polypeptide and a hydrophilic polymer in combination with an oxidizing agent, reducing agent, or ammonia has a “hair improvement effect” including, but not limited to, smoothing, thickening, repair, increased flexibility, and coating.

The term “keratin” refers to a family of fibrous structural proteins that are found in the hair, skin, nails, claws and hooves of mammals (α-keratins) and in the scales, claws and shells of reptiles, the feathers, beaks, and claws of birds and the quills of porcupines (β-keratins). Alpha-keratins are also known as cytokeratins and are further subdivided into soft α-keratins (epithelial cytokeratins) and hard α-keratins (trichocyte keratins). All keratins are heteropolymers of type I and type II keratins. The HUGO Gene Nomenclature Committee identifies the following as known keratin polypeptide encoding genes: KRT1, KRT2, KRT3, KRT4, KRT5, KRT6A, KRT6B, KRT6C, KRT7, KRT8, KRT9, KRT10, KRT12, KRT13, KRT14, KRT15, KRT16, KRT17, KRT18, KRT19, KRT20, KRT23, KRT24, KRT25, KRT26, KRT27, KRT28, KRT31, KRT32, KRT33A, KRT33B, KRT34, KRT35, KRT36, KRT37, KRT38, KRT39, KRT40, KRT71, KRT72, KRT73, KRT74, KRT75, KRT76, KRT77, KRT78, KRT79, KRT80, KRT81, KRT82, KRT83, KRT84, KRT85, KRT86, KRT222. Accordingly, a keratin polypeptide of the present invention may be encoded by one or more genes selected from KRT1, KRT2, KRT3, KRT4, KRT5, KRT6A, KRT6B, KRT6C, KRT7, KRT8, KRT9, KRT10, KRT12, KRT13, KRT14, KRT15, KRT16, KRT17, KRT18, KRT19, KRT20, KRT23, KRT24, KRT25, KRT26, KRT27, KRT28, KRT31, KRT32, KRT33A, KRT33B, KRT34, KRT35, KRT36, KRT37, KRT38, KRT39, KRT40, KRT71, KRT72, KRT73, KRT74, KRT75, KRT76, KRT77, KRT78, KRT79, KRT80, KRT81, KRT82, KRT83, KRT84, KRT85, KRT86, and KRT222.

As used herein, the term “polypeptide” is used in its broadest sense to refer to a compound of two of more subunit amino acids, amino acid analogs, or peptidomimetics. The subunits may be linked by peptide bonds. In another embodiment, the subunits may be linked by other bonds, e.g., ester, ether, etc. As used herein, the term “amino acid” refers to either natural and/or unnatural or synthetic amino acids, including glycine and both the D or L optical isomers, and amino acid analogs and peptidomimetics. A peptide of three or more amino acids may be referred to as an oliogpeptide if the peptide chain is short, e.g., less than about 25 amino acids. The term “polypeptide” can include full-length naturally occurring proteins as well as functional fragments of those proteins. Protein fragments are functional when they achieve the desired result of the full-length protein in the context of the present invention. For example, a keratin fragment that is covalently bound to a hydrophilic polymer is encompassed by the methods herein when the keratin-hydrophilic polymer provides a hair improvement effect when applied to hair. A polypeptide can be derived from natural sources or synthetically prepared, including through the use of large-scale protein expression mechanisms. The polypeptides may be hydrolyzed or non-hydrolyzed, and in some embodiments, the polypeptides are non-hydrolyzed. In one aspect, hydrolyzed keratin polypeptide is used in the conjugate.

It should be understood that the keratin polypeptide can be naturally derived or synthetically prepared. The keratin polypeptide can be purified from various natural sources or commercially obtained. The keratin polypeptide can have a molecular weight from about 0.5 kDa to about 60 kDa, (such as 0.5 kDa to about 40 kDa, 0.5 kDa to about 30 kDa, 0.5 kDa to about 20 kDa, 0.5 kDa to about 10 kDa) or from about 2 kDa to about 6 kDa, or from about 20 kDa to about 40 kDa. In some embodiments, the keratin polypeptide is about 5 kDa. In some embodiments, the keratin polypeptide is about 45 kDa to about 55 kDa. In still other embodiments, the keratin polypeptide is about 52 kDa. Size exclusion chromatography may be used to find a range of molecular weights of polypeptides that may be useful herein.

As used herein, the term “hydrophilic polymer” includes polypeptides, carbohydrates, nucleic acids, poly(ethylene glycol), poly(oxazoline), poly(vinylpyrrolidone), poly(acrylamide), poly(acrylic acid), poly(allylamine), poly(hydroxyethyl methacrylate), poly(ethyleneimine), poly(vinylphosphonic acid), poly(vinyl sulfate), poly(vinylsulfonic acid), poly(vinyl alcohol), glycerol propoxylate, hydroxyethyl starch (HES). In one embodiment, the hydrophilic polymer is a synthetic hydrophilic polymer selected from the group consisting of: poly(ethylene glycol), poly(oxazoline), poly(vinylpyrrolidone), poly(acrylamide), poly(acrylic acid),poly(allylamine), poly(hydroxyethyl methacrylate), poly(ethyleneimine), poly(vinylphosphonic acid), poly(vinyl sulfate), poly(vinylsulfonic acid), poly(vinyl alcohol), glycerol propoxylate, and hydroxyethyl starch (HES). In some embodiments, the molecular weight of the hydrophilic polymer is between about 0.5 and about 80 kDa, between about 0.5 and about 40 kDa, or between about 0.5 and about 10 kDa. In some embodiments, the molecular weight of the hydrophilic polymer is between about 2 and about 10 kDa. In some embodiments, the molecular weight of the hydrophilic polymer is between about 20 and about 40 kDa. In one embodiment, the molecular weight of the hydrophilic polymer is about 5 kDa. In another embodiment, the molecular weight of the hydrophilic polymer is about 30 kDa.

In other or further embodiments, the hydrophilic polymer has a formula selected from:

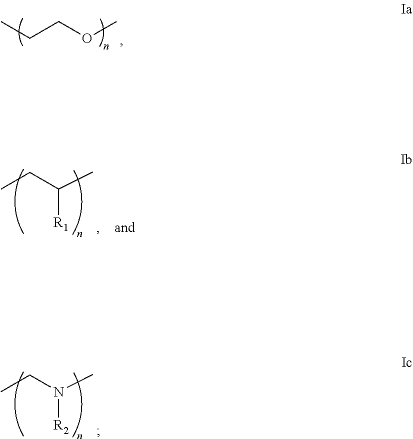

wherein R₁ is hydroxyl, carboxyl, ethylamine, 2-pyrrolidone, hydoxyethyl methacrylic acid, methylamine, phosphonic acid, sulfonic acid, hydroxyl, or SO₄⁻; wherein R₂ is hydrogen, methyloxazoline, ethyloxazoline, or propyloxazoline; and wherein “n” is between 1 and 1,500. In some embodiments, the “n” in formula Ia, Ib or Ic is between 1 and 1000, 1 and 500, 1 and 250, or 1 and 125. In some embodiments, the “n” in formula Ia, Ib or Ic is between about 100 and about 125. In still other embodiments, the “n” is about 114. In still further embodiments, the “n” in formula Ia, Ib or Ic may be selected from 1, 2, 3, 4, 5, 6, 7, 8, 9, 10, 11, 12, 13, 14, 15, 16, 17, 18, 19, 20, 21, 22, 23 and 24.

In yet another embodiment, the hydrophilic polymer is covalently bound to a keratin polypeptide and the hydrophilic polymer has a formula of:

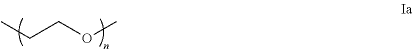

wherein n is between 1 and 1,500. In some embodiments, the “n” in formula Ia, Ib or Ic is between 1 and 1000, 1 and 500, 1 and 250, or 1 and 125. In some embodiments, the “n” in formula Ia, Ib or Ic is between 100 and 125. In still other embodiments, the “n” is about 114. In still further embodiments, the “n” in formula Ia, Ib or Ic may be selected from 1, 2, 3, 4, 5, 6, 7, 8, 9, 10, 11, 12, 13, 14, 15, 16, 17, 18, 19, 20, 21, 22, 23 and 24.

The compounds of formula Ia are known to those of skill in the art as polyethylene glycol, or PEG, compounds. There are a wide range of PEG compounds that can be used—these compounds can be linear or branched, reactive on one or both ends (mono- or bi-functional), or non-reactive in the traditional sense. The PEG compounds encompassed by the present invention are capable of being covalently bound to a polypeptide at reactive moiety on the polypeptide, or a polypeptide modified to include such a reactive moiety, however, the invention is not limited by the covalent attachment method. In some embodiments, the PEG compound is functionalized to allow reaction with the polypeptide. In some embodiments, the hydrophilic polymer is a PEG compound that covalently binds a polypeptide at a primary amine (e.g. a lysine residue). In other embodiments, the hydrophilic polymer is a PEG compound that covalently binds a polypeptide at a sulfhydryl (e.g. a cysteine residue).

Non-limiting examples of hydrophilic PEG polymers that fall within the present invention are MS(PEG)₄ (Thermo Scientific, Rockford, Ill., USA), MS(PEG)₈ (Thermo Scientific, Rockford, Ill., USA), MS(PEG)₁₂ (Thermo Scientific, Rockford, Ill., USA), MS(PEG)₂₄ (Thermo Scientific, Rockford, Ill., USA), TMS(PEG)₁₂ (Thermo Scientific, Rockford, Ill., USA), TMM(PEG)₁₂ (Thermo Scientific, Rockford, Ill., USA), MM(PEG)₁₂ (Thermo Scientific, Rockford, Ill., USA), MM(PEG)₂₄ (Thermo Scientific, Rockford, Ill., USA), and other N-hydroxysuccinimide functionalized PEGs, mPEG-Succinimidyl-Succinate (molecular weight of 2, 5, 10 20 or 30 kDa) (Laysan Bio., Inc., Arab, AL, USA), mPEG-Nitrophenyl carbonate (molecular weight of 2, 5, 10, 20, or 30 kDa) (Laysan Bio., Inc., Arab, AL, USA), and mPEG-Succinimidyl Glutarate (molecular weight of 2, 5, 10 20 or 30 kDa) (Laysan Bio., Inc., Arab, AL, USA). “Branched” polyethylene glycol compositions include TMS(PEG)₁₂, TMM(PEG)₁₂, and any other multi-armed polyethylene glycol compositions. In one embodiment, the hydrophilic polymer is an mPEG-Succinimidyl-Succinate polymer that is about 5 kDa and wherein “n” is about 114. The number after “(PEG)” in the foregoing examples of hydrophilic PEG polymers refers to the number of polyethylene glycol units. In some aspects, the polyethylene glycol compounds have terminal methyl groups. Some polyethylene glycol compounds allow modification of proteins that have sulfhydryl groups. For example, maleimide-activated polyethylene glycol compounds (e.g., MM(PEG)₁₂, MM(PEG)₂₄, etc.) may react specifically with free reduced sulfhydryl to form stable thioether bonds. MS(PEG)n or TMS(PEG)n compounds are amine reactive (e.g., MS(PEG)n or TMS(PEG)n).

TABLE 1

| Illustrative | Exemplary Functionally | |
|---|---|---|
| Functionally Reactive Moieties | Reactive Moieties on | |
| on Polypeptide/Polysaccharide Y | Hydrophilic Polymer X | Covalent Product Y-X |
| Y—COOH | HO—X | Y—C(═O)O—X |
| | (hydroxyl or activated forms | |
| | thereof (e.g., tresylate, mesylate | |
| | etc.)) | |
| Y—COOH | HS—X | Y—C(═O)S—X |
| Y—SH | (thiol) | Y—S—S—X |
| Y—SH | R′—S—S—X | Y—S—S—X |
| | (disulfide) | |
| Y—SH | (pyridyl)-S—S—X | Y—S—S—X |
| | (dithiopyridyl) | |
| Y—NH₂ | H(O═)C—X | Y—N═CH—X or |
| | aldehyde | Y—NH—CH₂—X |
| | | following reduction |
| Y—NH₂ | (HO)₂HC—X | Y—N═CH—X or |
| | aldehyde hydrate | Y—NH—CH₂—X |
| | | following reduction |
| Y—NH₂ | (R′O)₂CH—X or 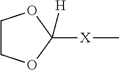 acetal | Y—N═CH—X or Y—NH—CH—X following reduction |
| Y—NH₂ | R′OCH(OH)—X or | Y—N═CH—X or |
| | hemiacetal | Y—NH—CH—X |
| | | following reduction |
| Y—NH₂ | R′(O═)C—X | Y—N═CR′—X or |
| | ketone | Y—NH—C(R′)H—X |
| | | following reduction |
| Y—NH₂ | (R′O)₂C(R′)—X or 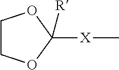 ketal | Y—N═C(R′)—X or Y—NH—C(R′)H—X following reduction |
| Y—NH₂ | R′OC(R′)(OH)—X | Y—N═C(R′)—X or |
| | hemiketal | Y—NH—C(R′)H—X |
| | | following reduction |
| Y—NH₂ | R′(S═)C—X | Y—N═C(R′)—X or |
| | ketone | Y—NH—C(R′)H—X |
| | thione (thioketone) | following reduction |
| Y—NH₂ | (R′O)(R′S)C(R′)—X or 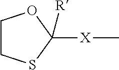 monothioketal | Y—N═C(R′)—X or Y—NH—C(R′)H—X following reduction |
| Y—NH₂ | R′SC(R′)(SH)—X or | Y—N═C(R′)—X or |
| | dithiohemiketal | Y—NH—C(R′)H—X |
| | | following reduction |
| Y—NH₂ | (R′S)₂C(R′)—X or 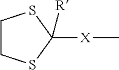 dithioketal | Y—N═C(R′)—X or Y—NH—C(R′)H—X following reduction |
| Y—SH Y—OH Y—COOH (anion) Y—NHR″ | 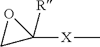 epoxide (oxirane) | Y—S—CH₂—C(OH)(R″)—X— Y—O—CH₂—C(OH)(R″)—X— Y—C(═O)O—CH₂—C(OH)(R″)—X— Y—NR″—CH₂—C(OH)(R″)—X— |
| Y—SH Y—OH Y—COOH (anion) Y—NHR″ | 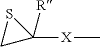 thioepoxide | Y—S—CH₂—C(SH)(R″)—X Y—O—CH₂—C(SH)(R″)—X— Y—C(═O)O—CH₂—C(SH)(R″)—X— Y—NR″—CH₂—C(SH)(R″)—X— |
| Y—SH | HO—(C═O)—X | Y—S—(C═O)—X |
| Y—OH | carboxyl | Y—O—(C═O)—X |
| Y—NHR″ | | Y—N(R″)—(C═O)—X |
| Y—SH | (alcohol)-(C═O)—X | Y—S—(C═O)—X |
| Y—OH | carboxylic acid ester | Y—O—(C═O)—X |
| Y—NHR″ | (alcohol indicates an esterified | Y—NR″—(C═O)—X |
| | suitable alcohol leaving group | |
| | e.g., p-nitrophenyl) | |
| Y—NH₂ | 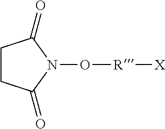 N-hydroxysuccinimide ester | Y—NH—R′′′—X |
| Y—SH | 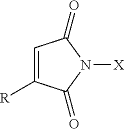 R = H, CH₃ | 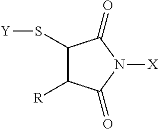 R = H, CH₃ |
| Y—NH₂ | 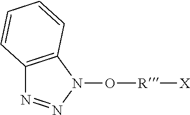 1-benzotriazole ester | Y—NH—R′′′—X |
| Y—NH₂ | CH₃—((CH₂)₁₋₃)—O(C═NH)—X | Y—NH—(C═NH)—X |
| | (imidoester) | (amidine) |
| Y—(C═NH)—O—((CH₂)₁₋₃)—CH₃ | H₂N—X | Y—(C═NH)—HN—X |
| (imidoester) | | (amidine) |
| Y—COOH | H₂N—X | Y—(C═O)—NH—X |
| Y—(C═O)—R″ | amine | Y—(R″)C═N—X or |
| | | Y—(R″)CH—NH—X |
| | | following reduction |
| Y—COOH | H₂N—(C═O)—NH—X | Y—(C═O)—NH—(C═O)—NH—X |
| Y—(C═O)—R″ | urea | Y—(R″)C═N—(C═O)—NH—X or |
| | | Y—(R″)CH—NH—(C═O)—NH—X |
| | | following reduction |
| Y—COOH | H₂N—(C═O)—O—X | Y—(C═O)—NH—(C═O)—O—X |
| Y—(C═O)—R″ | carbamate | Y—(R″)C═N—(C═O)—O—X or |
| | | Y—(R″)CH—NH—(C═O)—O—X |
| | | following reduction |
| Y—COOH | H₂N—(C═S)—NH—X | Y—(C═O)—NH—(C═S)—NH—X |
| Y—(C═O)—R″ | thiourea | Y—(R″)C═N—(C═S)—NH—X or |
| | | Y—(R″)CH—NH—(C═S)—NH—X |
| | | following reduction |
| Y—COOH | H₂N—(C═S)—O—X | Y—(C═O)—NH—(C═S)—O—X |
| Y—(C═O)—R″ | thiocarbamate | Y—(R″)C═N—(C═S)—O—X or |
| | | Y—(R″)CH—NH—(C═S)—O—X |
| | | following reduction |
| Y—(C═O)—R″ | H₂N—HN—X | Y—(R″)C═N—HN—X |
| | | hydrazone |
| Y—NH—NH₂ | R″—(O═C)—X | Y—NH—N═C(R″)—X |
| | | hydrazone |
| Y—NH₂ | O═C═N—X | Y—NH—(C═O)—NH—X |
| Y—OH | isocyanate | Y—O—(C═O)—NH—X |
| Y—NH₂ | S═C═N—X | Y—NH—(C═S)—NH—X |
| Y—OH | isothiocyanate | Y—O—(C═S)—NH—X |
| Y—SH | H₂C═CH—(C═O)—X or | Y—S—CH₂CH₂—(C═O)—X |
| | H₂C═C(CH₃)—(C═O)—X | Y—S—CH₂—CH(CH₃)—(C═O)—X |
| | alpha-beta unsubstituted carbonyls | |
| Y—SH | H₂C═CH—(C═O)O—X | Y—S—CH₂CH₂—(C═O)O—X |
| | alpha-beta unsubstituted carboxyl | |
| Y—SH | H₂C═C(CH₃)—(C═O)—O—X | Y—S—CH₂CH(CH₃)—(C═O)O—X |
| | alpha-beta unsubstituted carboxyls | |
| | (methacrylates) | |
| Y—SH | H₂C═CH—(C═O)NH—X | Y—S—CH₂CH₂—(C═O)NH—X |
| | alpha-beta unsubstituted amides | |
| | (acrylamides) | |
| Y—SH | vinylpyridine-X | Y—S—CH₂—CH₂-(pyridyl)-X |
| | (2- or 4-vinylpyridine) | |
| Y—SH | H₂C═CH—SO₂—X | Y—S—H₂C—CH₂—SO₂—X |
| | (vinyl sulfone) | |
| Y—SH | ClH₂C—CH₂—SO₂—L | Y—S—H₂C—CH₂—SO₂—X |
| | (chloroethyl sulfone) | |
| Y—SH | (halogen)-CH₂—(C═O)—O—X | Y—S—CH₂—(C═O)—O—X |
| | (halogen)-CH₂—(C═O)—NH—X | Y—S—CH₂—(C═O)—NH—X |
| | (halogen)-CH₂—(C═O)—X | Y—S—CH₂—(C═O)—X |
| | (halogen is preferably I or Br) | |
| Y—O(C═O)—CH₂-(halogen) | HS—X | Y—O(C═O)—CH₂—S—X |
| Y—NH(C═O)—CH₂-(halogen) | | Y—NH(C═O)—CH₂—S—X |
| Y—(C═O)—CH₂-(halogen) | | Y—(C═O)—CH₂—S—X |
| (halogen is preferably I or Br) | | |
| Y—SH | (halogen)-CH₂(C═O)O—X | Y—S—CH₂(C═O)O—X |
| | (halogen)-CH₂(C═O)NH—X | Y—S—CH₂(C═O)NH—X |
| | (halogen)-CH₂(C═O)—X | Y—S—CH₂(C═O)—X |
| | (halogen is preferably I or Br) | |
| Y—N₃ | HC≡C—X | 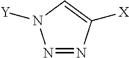 |
| Y—N₃ | 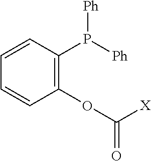 | 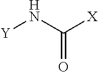 |
| Y—N₃ | 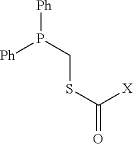 | 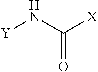 |
| Y—SH | 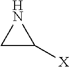 | 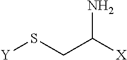 |
| Y—NH₂ | (F₅—Ph)—OC(O)—X | Y—NH—C(O)—X |

R′ is C₁₋₆ alkyl, C₃₋₆ cycloalkyl, or an aryl group having 5-8 endocyclic atoms;
R″ is H, C₁₋₆ alkyl, C₃₋₆ cycloalkyl, or an aryl group having 5-8 endocyclic atoms;
R′″ is a carbonyl derivative *—(C═O)—, *—(C═O)—(CH₂)₁₋₈—*—(C═O)—(CH₂)₁₋₈—S—S—, *—(C═O)—(CH₂)₁₋₈—(C═O)—O—, *—(C═O)—(CH₂)₁₋₈—O—(C═O)—, *—(C═O)—(CH₂)₁₋₈—(C═O)—NH—, or *—(C═O)—(CH₂)₁₋₈—NH—(C═O)—, or alternatively, R′″ is a carbonyl derivative of the form *—(C═O)—O—(CH₂)₁₋₈—S—S—, *—(C═O)—O—(CH₂)₁₋₈—(C═O)—O—, *—(C═O)—O—(CH₂)₁₋₈—(C═O)—O—(CH₂)₁₋₈— *—(C═O)—O—(CH₂)₁₋₈—O—(C═O)—, *—(C═O)—O—(CH₂)₁₋₈—(C═O)—NH—, or *—(C═O)—O—(CH₂)₁₋₈—NH—(C═O)—, where “*” indicates the point of attachment to succinimidyl or benzotriazolyl groups;
X and Y are each the polypeptide and the hydrophilic polymer, respectively.

Examples of PEG compounds useful in the conjugate herein include

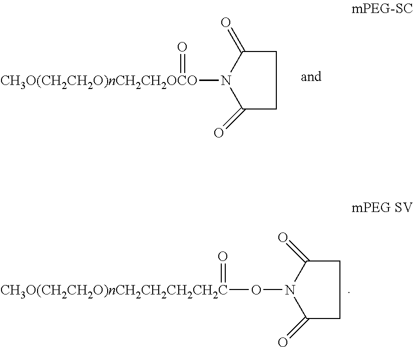

Permanent hair color generally contains ammonia and must be mixed with a developer or oxidizing agent in order to permanently change hair color. Ammonia, in permanent hair color, is used to open the cuticle layer so that the developer and colorants together penetrate into the cortex. Ammonia may also be used to allow the conjugate to enter the cuticle layer.

Permanent hair dyes contain a primary intermediate such as:

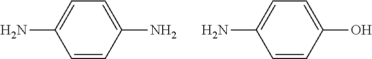 [SMILES: Nc1ccc(N)cc1 ; Nc1ccc(O)cc1]

These are often p-diamines or p-aminophenols. They are oxidized by hydrogen peroxide to give reactive species which then react with couplers such as: resorcinol, m-aminophenol, 2-methyl-5-aminophenol, p-phenylenediamine, 2,4-diaminoanisole, 1,5-dihydroxynaphthalene, 4-methoxy-3-aminophenol, 2,4-di-aminophenoxyethanol, m-diethylaminophenol, p-amino-o-cresol to produce dyes. For example, using

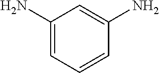

as the coupler below ([O] is a peroxide):

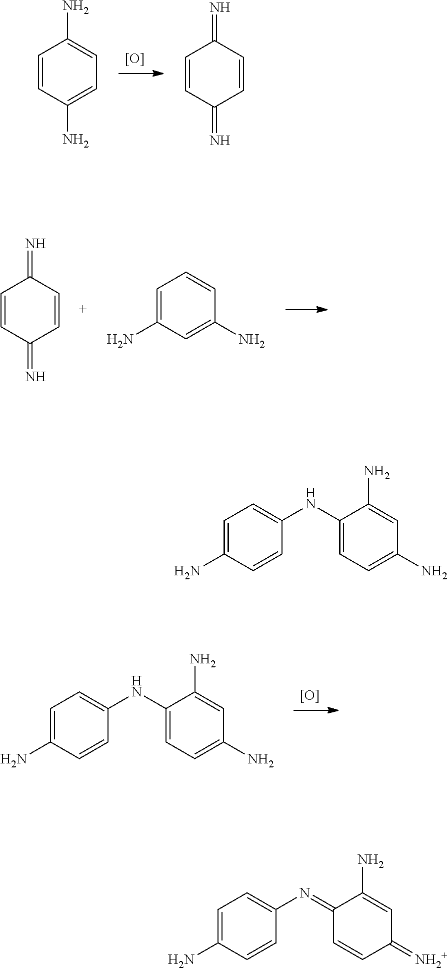

The final hair dye is locked inside hair during processing.

Without being bound by theory, it is believed that during the oxidation step, the primary intermediate becomes a reactive species that couples to the conjugate H₂N-[HK-Peg], where K is the hydrolyzed keratin polypeptide, and the Peg is polyethylene glycol below. It is believed that a primary intermediate, such as p-diamine, forms a reactive species upon oxidation and covalently binds through an amine group to a conjugate as follows:

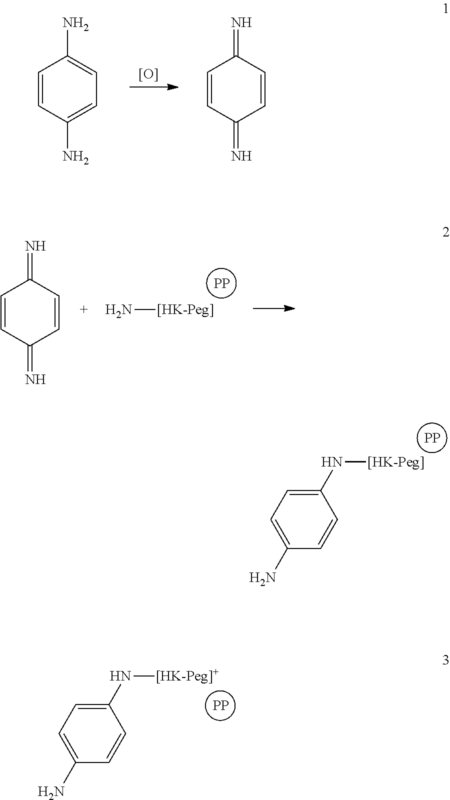

Without being bound by theory, the resulting compound may attach to hair in various ways. In one theory, the resulting compound may be attached to the coupler and then passes into the cortex underneath the cuticle and locks into hair as with the dye. In another theory, the resulting compound forms another reactive species with the remaining oxidizing agent that binds to a coupler. Yet in another theory, the resulting compound may form a disulfide bond with hair. In another theory, the resulting compound may form hydrogen bonds to the hair.

In addition, it is surprising that the conjugate, when used in the methods herein or added to the combinations herein, did not slow down the processing time of the various coloring hair bleaching reactions in comparison to when Olaplex (marketed to rebuild broken hair bonds) was used in the same processes.

Permanent hair straightening or perming techniques use harsher methods, with chemicals, to break the disulfide bonds and reform them. The disulfide bonds cannot be broken apart by oxidizing agents, which may be acids, but can be broken apart by strong reducing agents, which are bases. Alkaline solutions, therefore, are applied to hair to break apart the disulfide bonds. The hair is then held straight or wrapped in curlers, and acidic solutions are applied to the hair.

The acid neutralizes the base, stopping the disulfide bond cleavage reaction and allowing the disulfide bonds to reform in new positions. Although all chemical hair-straightening techniques have this same basis, each has a slightly different mode of action on the hair. Alkaline hydroxides are the harshest and most rapid agents for breaking the disulfide bonds; usually, either sodium hydroxide or calcium hydroxide is used for this purpose. Along with breaking the disulfide bonds, these solutions also cause the hair to swell. Since they are so harsh, these chemicals should not be left in the hair for more than 10 minutes, as they can cause severe damage to the hair and scalp if not used properly. Hair is usually treated with a cream to protect it from the caustic effects of these chemicals before applying the alkaline hydroxides. Thoroughly washing off the solutions allows the reaction to stop and the disulfide bonds to reform, sealing the hair in its new, straightened position.

Solutions containing thioglycolate are safer to use, such as ammonium thioglycolate. Thioglycolate is much less harsh than alkaline hydroxides, it is a more popular method for hair straightening or perming (curling). Thioglycolate contains a thiol (—SH) group, and dissociation of the hydrogen atom from the sulfur creates a thiolate (—S—) ion. Thiolate ions are good reducing agents and help break the disulfide bonds. This method generally takes much longer than alkaline hydroxides. Also, a strong oxidizing agent, such as hydrogen peroxide, has to be applied after thioglycolate treatment in order to stop the reaction and help the bonds reform. Ammonium thioglycolate can break disulfide bonds and releases ammonia which loosens the hair and allows the glycolate to sleep through the hair:

Keratin-S—S-keratin+2HS—CH₂CO₂NH₄—>—HO₂CH₂CS—SCH₂CO₂H+2NH₃+2HS-keratin

The hair can be tied around curlers or shaped and then strengthened so the style becomes permanent for this common oxidation agent is applied, which contains hydrogen peroxide which reconstitutes the disulfide bonds:

2Keratin-SH+H₂O₂->Keratin-SS-keratin+2H₂O

Another method is the use of bisulphate-containing solutions. These, too, can act as reducing agents. They work slightly better than thioglycolates and are safer than alkaline hydroxides.

Another hair processing technique is to apply lye soap which contains about 5-10% of sodium hydroxide which breaks the disulfide bonds. The hair loosens up causing curls to disappear. The sodium hydroxide is removed upon washing hair. It is believed, without being bound by theory, that the disulfide bonds join one sulfur atom on one polypeptide on a subject's hair to a sulfur atom on a polypeptide chain of the e.g., hydrolyzed keratin polypeptide in the conjugate.

Without being bound by theory, the conjugate herein when combined with the oxidizing agent during the neutralizing phase, can attach to keratin in a subject's hair (after the removal of the hydrogen atoms from the sulfur atoms during the application of a reducing agent) and reforms the disulfide bonds. It is believed that the disulfide bonds join one sulfur atom on one polypeptide on a subject's hair to a sulfur atom on a polypeptide chain of the keratin in the conjugate.

The polypeptide-hydrophilic polymer and polysaccharide-hydrophilic polymer compositions described above and elsewhere herein are applied to the hair of a subject for have a hair improvement effect.

Those of skill in the art will understand that the specific level of administration and frequency of usage for any particular subject can be varied and will depend upon a variety of factors, including the activity of the specific compound employed, the metabolic stability and length of action of that compound, age, the hair texture and length of the subject's hair, the mode and time of administration, and severity of the particular hair damage. In some embodiments, the keratin-hydrophilic polymer combinations are applied to the hair when bleaching, coloring or perming hair, at a frequency of application such as once about every 3 weeks, once about every 4 weeks, once about every 5 weeks, once about every 6 weeks, once about every 7 weeks, about once or twice per month, about once per 2 months, about once per 3 months, about once per 4 months, or longer as needed.

Administration of a polypeptide-hydrophilic polymer and/or polysaccharide-hydrophilic polymer and oxidizing agent combination to the hair in an effective amount can result in a hair improvement effect.

U.S. patent application Ser. No. 11/824,400 refers to hair treatment compositions comprising a multi-arm polyethylene glycol derivative that contains two or more functional groups that may be covalently bound to hair.

In some aspects, the hydrophilic polymer such as polyethylene glycol used in the conjugates herein only attach to the polysaccharide or polypeptide such as hydrolyzed keratin polypeptide on one end of the polysaccharide or polypeptide. The other end or ends of the polysaccharide or polypeptide does not have a hydrophilic polymer such as polyethylene glycol compound covalently bound thereto. In some aspects, if the hydrophilic polymer such as polyethylene glycol compound is linear, only one end covalently binds to polysaccharide or polypeptide such as hydrolyzed keratin polypeptide. In some aspects, if the hydrophilic polymer such as polyethylene glycol compound is branched, only one end has a functional group that covalently binds to a polysaccharide or polypeptide such as keratin or hydrolyzed keratin polypeptide. Using a hydrophilic polymer such as a polyethylene glycol compound that has one functional group that is capable of covalently binding to a polysaccharide or polypeptide such as hydrolyzed keratin polypeptide (e.g., through an amine), allows the resulting conjugate to be water-soluble and not cross-linked. In some aspects, the hydrophilic polymer such as a polyethylene glycol compound has one functional group that can covalently bind to a polysaccharide or polypeptide such as hydrolyzed keratin polypeptide during the reaction to make the conjugate. When two or more functional groups are present on a polyethylene glycol compound, cross-linking may occur that may form an insoluble or less soluble conjugate. In some aspects herein, the combinations herein are water soluble and are well suited to be combined with a water soluble conjugate.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims. All publications, patents and patent applications cited herein are hereby incorporated by reference in their entirety for all purposes.

### EXAMPLES

U.S. patent application Ser. No. 14/419,283 and WO2015/187951 include various methods of making the conjugate that are incorporated herein by reference in their entireties.

### Example 1

### Preparation of Keratin-Polvethylene Glycol Composition

Keratin-polyethylene glycol compositions were prepared using the following equipment and materials:
Vortexer
Shaker
Pipettes (1000 micro liter)
Syringes (1 cc)
1.50 ml tubes
Weighing balance Mettler Malani Ser #172447
Fume hood
DPBA-Dulbecco's phosphate buffered saline (1×) Cat #14190-144, lot #901681, Gibco Invitrogen.
N,N-dimethyl formamide, Cat # D119-500, lot #963760
MS(PEG)4; [MS4] Cat #22341, lot # MI162217, Thermo Scientific (Pierce)
MS (PEG)12; [MS12] Cat #22685, lot # MI162218, Thermo Scientific (Pierce)
MS (PEG)24; [MS24] Cat #22687, lot # MH161869, Thermo Scientific (Pierce)
TMS (PEG)12; [TMS12] Cat #22421, lot # MD157106, Thermo Scientific (Pierce)
Keratin [K] Cat # K3030, lot #1AG0185, Spectrum hydrolyzed keratin polypeptide.

Using the above described materials and equipment, 10 mg of Keratin powder was placed in 1.5 mL centrifuge tubes (5 in number). The five samples were labeled K, K+MS4, K+MS12, K+MS24, and K+TMS12. One milliliter of phosphate buffered saline (PBS) was added to each of the 10 mg keratin samples and let shake for 45 minutes. Then 15 μl of about 250 mM MS4, MS12, MS24 and TMS12 pegylation stock solutions were added to each keratin sample tube and placed on a shaker for 30 minutes. The pegylation stock solutions were prepared using the following calculations:
Keratin M.W.=52.5 kDa
1 mL Conjugated protein ×10 mg/1 mL×1/52500×20/1=0.003809 mmol pegylated reagent. Then 0.003809×1,000,000 μL/L×L/250 mmol=15.23.
About 15 μL of 250 mM pegylation reagent stock. Materials were equilibrated at room temperature.
MS4 1.1 mL DMF to 100 mg of stock MS4
MS12 485 μl DMF to 100 mg of stock MS12
MS24 230 μl DMF to 100 mg of stock MS24
TMS12 65 μl DMF to 100 mg of stock TMS12

The resultant solutions were stored in a freezer box with desiccant and returned to −20° C. freezer until used.

### Example 2

Preparation of Keratin-Polyethylene Glycol (SVA) Compositions
Keratin-polyethylene glycol compositions were prepared using the following materials and equipment:
1. Pipettes
2. Syringes
3. 1.5 ml centrifuge tubes
4. Weighing balance Mettler
5. Methoxy-Poly (Ethylene Glycol)-Succinimidyl Valerate lot #120-176; Laysan Bio Inc. 5000 MW (SVA PEG) (5k SVA PEG)
6. DPBS-Dulbecco's phosphate buffered saline cat #14190, lot #1048427, Gibco
7. Keratin Cat # K3030, lot #1AG0185, Spectrum

Using the above-described materials and equipment, reaction samples were prepared having different ratios of keratin polypeptide and SVA PEG polymer (1:1, 1:0.3, 1:0.1). Samples were also prepared that contained only SVA PEG polymer as prepared according to this example (SVA Neat), only TMS-12 polymer as prepared according to Example 1 (TMS-12 Neat), only MS-24 polymer as prepared according to Example 1 (MS-24 Neat), and only PBS (Solution Neat). These samples were prepared as follows:
In 1.5 ml centrifuge tubes 1 ml of DPBS was added. Sample tubes were labeled as follows:
1. SVA Neat=weighted out 10 mg of SVA PEG and added to 1 ml D-PBS.
2. K:P; 1:1=weighted out 50 mg of SVA PEG and added to 1 ml D-PBS+5 mg keratin
3. K:P; 1:0.1=weighted out 10 mg of SVA PEG and added to 1 ml D-PBS+10 mg Keratin
4. K:P; 1:0.3=weighted out 30 mg of SVA PEG and added to 1 ml D-PBS+10 mg Keratin
5. PBS neat=1 ml of D-PBS
6. TMS 12 neat=65 μi of TMS-12 added to 1 ml D-PBS
7. MS24 neat=230 μi of MS24 added to 1 ml D-PBS [0061] Different amounts [0, 5, 10 mg] of keratin were then added to 1 ml D-PBS from and shaken by hand to solubilize. Varying amount of SVA PEG were weighted out and added to the PBS-keratin solutions. The SVA PEG amounts ranged from 10 to 50 mg. Tubes were then shaken by hand and sealed with parafilm.

For the MS24 neat sample, 230 i of stock prepared according to Example 1 was added to 1 ml D-PBS. For the TMS12 neat sample 65 i of stock prepared according to Example 1 was added to 1 mL D-PBS.

### Example 3

### Preparation of Keratin-Polyethylene Glycol (SC) Compositions

Keratin-polyethylene glycol (SC) compositions were prepared in a similar fashion to the (SVA) compositions in Example 2, but using mPEG-SC instead of SVA PEG.

### Example 4

### Administration of a Keratin-Polvethylene Glycol Composition to the Hair

[01] The keratin-polyethylene glycol composition described in Example 3 was administered to the hair as described below (Healthilocks serum solution).

Healthilocks serum solution (or “Healthilocks”) was added directly to the bleach solution (or hair coloring solution) of 1 gram per application. The stylist applied the bleach or hair dye solution to the hair of the client and waited the appropriate amount of time for the hair colorant or bleach solution to take effect. The hair was then rinsed with water and then dried and styled. The observations on the hair is that there was a better shine as well as feel of softness and structural strength to the hair after the process with Healthilocks versus without. Note that for other products of typical nature and application there is a diluting effect and therefore a stylist is required to use a stronger solution or bleach or colorant in order to observe similar results to Healthilocks. The stronger solution is a limitation of these products, but not for Healthilocks which doesn't affect the solution strength and is safer. These other products make the process use harsher chemicals on the scalp and skin which leads to burns and other adverse issues for short and long-term use for the client and stylist. There is also an environmental impact to these products which contaminate with the additional materials and stronger solution. Healthilocks does not have this problem and is gentle and safe and keeps the current strength and materials to their safer conditions and less of an environmental impact.

Stylist 1.

The concentration of bleach was ipart powder bleach to 2 parts 20 vol. I left the bleach on previously lightened hair for 10-20 minutes to get it white. White hair is a big hair trend now so this came in great. The results were compared with Olaplex No. 1 Bond Multiplier (Water, Bis-Aminopropyl Diglycol Dimaleate, Phenoxyethanol, Sodium Benzoate).

The Healthilocks serum solution is better because it didn't slow down the processing time like Olaplex, but still helped preserve the integrity of the hair far more than without adding Healthilocks. It had a similar result to Olaplex in the sense that it keeps hair softer with less breakage than if no Healthilocks or Olaplex was used.

Stylist 2

Used 1 gram of Healthilocks serum solution for each of the treatments for coloring. The colors that were used for client 1 was red/auburn hair and for client 2 was brown/brunette. Both showed great results and full coloring of the hair without lightening as well as full coloring of grey. Softer hair, shinier and strong hair was observed.

### Example 2

Healthilocks was added to the neutralizing of a perming solution

What is claimed is:

1. A hair treatment method comprising
a) breaking a disulfide bond in a subject's hair to form a sulfide group on hair;
b) binding the sulfide group on hair with a sulfide group on a polypeptide such as a hydrolyzed keratin polypeptide in a conjugate, wherein the polypeptide is covalently bound to a hydrophilic polymer such as a polyethylene glycol compound in the conjugate.

2. A hair treatment method comprising
binding a conjugate to the subject's hair, wherein polysaccharide or polypeptide such as a hydrolyzed keratin polypeptide is covalently bound to a hydrophilic polymer such as a polyethylene glycol compound in the conjugate, without the use of heat.

3. A hair treatment method comprising mixing
a) a conjugate comprising a polysaccharide or a polypeptide such as a hydrolyzed keratin polypeptide that covalently binds to hydrophilic polymer such as a polyethylene glycol to form the conjugate, and
b) an oxidizing agent;
to form a mixture;
applying the mixture to a subject's hair.

4. The hair treatment method of claim 3
wherein the oxidizing agent comprises hydrogen peroxide.

5. The hair treatment method of claim 3 wherein the applying step occurs after a reducing agent is applied to a subject's hair.

6. The hair treatment method of claim 5 wherein the oxidizing agent is a neutralizing agent that neutralizes the reducing agent.

7. The hair treatment method of claim 3 wherein the mixture further comprises a dye.

8. The hair treatment method of claim 3 wherein the mixture comprises a reducing agent.

9. The hair treatment method of claim 3, wherein the conjugate binds to hair without the use of heat.

10. A hair treatment method comprising mixing
a) a conjugate comprising a polysaccharide or a polypeptide such as a hydrolyzed keratin polypeptide that covalently binds to hydrophilic polymer such as a polyethylene glycol to form the conjugate, and
b) a reducing agent;
to form a mixture;
applying the mixture to a subject's hair.

11. A hair treatment method comprising mixing
a) a conjugate comprising a polysaccharide or a polypeptide such as a hydrolyzed keratin polypeptide that covalently binds to hydrophilic polymer such as a polyethylene glycol to form the conjugate, and
b) ammonia;
to form a mixture;
applying the mixture to a subject's hair.

12. A hair treatment intermediate comprising a p-diamine or p-aminophenol that covalently binds through an amine group to a conjugate,
wherein the conjugate comprises a polysaccharide or a polypeptide such as a hydrolyzed keratin polypeptide that covalently binds to a hydrophilic polymer such as a polyethylene glycol to form the conjugate.

13. A hair treatment combination comprising
a conjugate comprising a polysaccharide or a polypeptide such as a hydrolyzed keratin polypeptide that covalently binds to hydrophilic polymer such as a polyethylene glycol to form the conjugate, and
ammonia.

14. A hair treatment combination comprising
a conjugate comprising a polysaccharide or a polypeptide such as a hydrolyzed keratin polypeptide that covalently binds to hydrophilic polymer such as a polyethylene glycol to form the conjugate, and
a reducing agent.

15. A hair treatment combination comprising
a conjugate comprising a polysaccharide or a polypeptide such as a hydrolyzed keratin polypeptide that covalently binds to hydrophilic polymer such as a polyethylene glycol to form the conjugate, and
an oxidizing agent.
